(19) United States
(12) Patent Application Publication
(10) Pub. No.: US 2005/0089484 A1
(43) Pub. Date: Apr. 28, 2005

(54) Sunless tanning products and processes

(76) Inventors: John J. McCook, Frisco, TX (US); Philip J. Gordon, Plano, TX (US); D. Craig Woodward, Plano, TX (US)

Correspondence Address: THEODORE JAY;APT 600, 16 N. CHATSWORTH AVE., LARCHMONT, NY, 10538, US

(21) Appl. No.: 10/694,972

(22) Filed: Oct. 28, 2003

(51) Int. Cl. A61K 7/16; A61K 7/42

(52) U.S. Cl. 424/59

(57) ABSTRACT

Sunless tanning spray compositions contain 1,2-Pentanediol, to produce low odor and long-lasting tans.

### CROSS REFERENCE TO CO-PENDING APPLICATIONS

The present application is related to two co-pending applications of John McCook et al, Ser. No. 10/024,822 entitled SUNLESS TANNING PRODUCTS AND PROCESSES, filed on Dec. 18, 2001 and Ser. No. 10/382,868 of like title, filed on Mar. 5, 2003.

### FIELD OF THE INVENTION

The present invention describes novel sunless tanning spray compositions containing pentylene glycol (1,2-pentanediol) for use in automated spray applications. These compositions promote an odorless and long-lasting tan.

### BACKGROUND OF THE INVENTION

Sunless tanning, also called self-tanning, is the ability to impart a tan to fair or light skin without the use of sunlight. In order to achieve a tanned look or otherwise darken their skin, individuals can expose their skin to sunlight or a source of simulated sunlight, e.g., a solar simulator or ultraviolet lamps. For many individuals, such exposure will stimulate formation of new melanin pigment and the retention of increased amount of melanin in the epidermis and produce a darkened skin color. However, some individuals find that such exposure does not produce the desired melanin formation and as a result the desired tan is not obtained. It is also well known that, as light skin humans age, the ability to produce melanin through ultraviolet light stimulation diminishes significantly. Additionally, exposure to the sun or a source of ultraviolet radiation can have deleterious effects for many individuals and can, in fact, cause sunburn, skin blistering, premature skin aging or skin cancer. Self-tanning or sunless tanning compositions offer a safe alternative and enable these individuals to obtain the desired tanned look.

Commercial formulations, using dihydroxyacetone [DHA], or DHA in combination with other reducing sugars such as 1,3,4-trihydroxy-2-butanone (erythrulose), applied locally to the skin, were developed for this purpose.

Typical sunless tanning preparations sold to the consumer are in the form of a cream, lotion, gel or aerosol foam or spray. Additionally, within the last few years, indoor tanning salons have begun to offer automated sunless tanning spray applications as a safe alternative to UV tanning beds. These sunless tanning sprays are applied either in an enclosed booth or with a hand-held spray apparatus and involve the pressurized application of a sunless tanning solution containing DHA or combinations of DHA and erythrulose and are typically delivered over the entire body in the form of a mist.

Sunless tanning booth operations are described in U.S. Pat. No. 6,387,081; “Misting apparatus for application of coating materials to skin surface”. Other US patents by Laughlin (U.S. Pat. Nos. 5,922,333; 6,199,557; 6,446,635; 6,474,343; 6,439,243, 6,431,180; and 6,305,384) by Parker (U.S. Pat. Nos. 6,443,164 and 6,302,122) and others describe automated spray systems for coating human skin with various cosmetic compositions including self-tanning compositions.

Typically the sunless tanning solutions used in these automated sunless tanning spray systems utilize relatively high levels of DHA (7-12%) versus the typical packaged sunless tanning creams, lotions, foams and sprays (3-7% DHA) sold in various retail outlets. Moreover, the automated sunless tanning spray systems typically deliver a mist of several ounces of sunless tanning solution in one misting session; much more sunless tanning product than typically would be self-applied by a consumer of a packaged sunless tanning product in any single application.

Sunless tanning booth sprays can coat the entire body with a light mist in one minute or less. Hand-held automated sunless tanning sprays utilizing an airbrush technique require several minutes to cover large areas of exposed skin. These pressurized spray systems dry quickly and produce a natural-looking tan. The spray booth systems, in particular, avoid the need for a second person to apply a sunless tanning cream, lotion, or foam product too hard to reach areas of the body. Additionally, the sunless tanning booth sprays and hand-held pressurized sprays are “simple” solutions of DHA without the need for oils, emulsifiers, surfactants, polymers, and other stabilizers that can result in greasiness, stickiness, and longer drying times. This fast drying characteristic of these sunless tanning automated misting systems can be an advantage over conventional sunless tanning creams, lotions, gels, and foams.

Complete drying of the sunless tanning formulation is necessary to avoid staining of clothing. Conventional sunless tanning products caution the consumer to wait 15 minutes or more until the sunless tanning product applied is completely dry before dressing or contact with clothing. Sunless tanning solutions applied via automated misting systems are dry within one-two minutes after application.

Notwithstanding the above mentioned advantages for automated sunless tanning spray systems, both these spray formulations and typical sunless tanning creams, lotions, gels, foams, and sprays can develop an off odor or malodor. This malodor is described as a “burnt sugar” or “burnt caramel” odor and is traceable to the DHA component of the sunless tanning compositions and DHA reaction with components of the skin. Additionally, many consumers who have developed a sunless tan with the automated spray formulations have expressed the desire for a longer lasting tan. Consumers report that the sunless tan that develops from automated misting systems lasts 3-5 days, very similar to typical sunless tanning products applied by hand. Nevertheless, consumers desire a sunless tan that lasts up to 7 days' for both convenience and cost reasons.

It is the object of this invention to provide new sunless tanning formulation technology to significantly reduce or eliminate sunless tanning formulation malodor and to promote a longer lasting tan. This technology has been shown to be particularly useful for sunless tanning solutions containing DHA as used in automated spray systems.

### SUMMARY OF THE INVENTION

Patent application Ser. No. 10/382686, filed Mar. 5, 2003 demonstrates that sunless tanning compositions are substantially improved by adding methylsulfonyl methane [MSM] or MSM and certain humectants or penetrants to dihydroxyacetone [DHA].

The preferred humectants or penetrants cited in the above patent application included 1,2-pentanediol, propylene glycol, 1,3-butylene glycol, 2-methyl-1, 3-propanediol, isopentyl diol, hexylene glycol, dimethyl isosorbide, or ethoxydiglycol. Pentylene glycol (1,2-pentanediol) was found to give the best results over the other humectants and penetrants when combined with MSM and DHA. Further experimentation has shown that cosmetic compositions containing pentylene glycol without the addition of MSM will produce a virtually odorless, longer lasting sunless tan. This is an unexpected result with pentylene glycol in sunless tanning formulations.

U.S. Pat. No. 6,214,322 for “Self-Tanning Composition Containing Carmine” assigned to Neutrogena Corp. mentions 1,2-pentanediol as an example of a humectant (along with glycerin, sorbitol, and other glycols) that may be included in the DHA containing self-tanning formulations cited. No mention is made of the inclusion of pentylene glycol to reduce odor or to extend the tanning response.

U.S. Pat. No. 6,113,888 for “Self-Tanning Mousse” assigned to Neutrogena also mentions pentylene glycol (1,2-pentanediol) as one of several humectants that may be included in the self-tanning compositions without any reference to the odor neutralizing or tan enhancing properties discovered by the applicants. Several other recent patents for personal or human use of products containing 1,2-pentanediol cite the use of this compound as a preservative, an antibacterial, an antifungal, or as an insect repellant.

No published prior art has suggested pentylene glycol as a unique agent to preferentially enhance the tanning response of DHA or as an ingredient to reduce or eliminate the potential malodor from DHA.

Various experiments conducted by the applicants have demonstrated that the addition of pentylene glycol to DHA is superior to all other glycols tested in it's ability to produce a darker, streak-free and longer-lasting tan. Additionally, the addition of pentylene glycol to sunless tanning compositions containing DHA reduces the potential for any malodor development. Sunless tanning compositions containing pentylene glycol are particularly useful in automated sunless tanning spray systems. However, sunless tanning creams, lotions, gel, and mouse compositions containing pentylene glycol should also demonstrate this enhanced performance.

The following formulations and the results of testing the formulations detailed in Tables 1 thru 10 compare the tanning response of pentylene glycol (1,2-pentanediol; Hydolite-5; Dragoco) and DHA versus the tanning response of DHA and several humectants and penetrants found to give a superior tanning response with DHA in previous testing outlined in U.S. application Ser. No. 10/382686, filed Mar. 5, 2003. In typical commercial sunless tanning products, the various humectants or penetrants are used at concentrations ranging from 1-5%. The following experiments compared the tanning response of the DHA solutions with glycols/penetrants in the range of 1-20%. All formulations were aqueous solutions made with deionized water and adjusted to a pH of 3.0-3.5 with a solution of citric acid (25%) to maximize the stability and tanning response of DHA. The solutions contained no non-water soluble emollients, emulsifiers or other ingredients that could inhibit skin penetration or slow drying time of the aqueous solution when applied as a simple solution or as a spray in an automated misting system.

Tables 1-5 compared the ability of pentylene glycol, 1,3-butylene glycol, hexylene glycol, DMI and propylene glycol to enhance the tanning response of DHA. No positive control (e.g., DHA & water) was included since previous experiments clearly documented those glycols will enhance the DHA tanning response versus DHA/water solutions.

Table 1 shows a first series of glycol humectants and penetrant materials used at a concentration of 1% w/w in combination with 5% DHA w/w; the amount of DHA typically found in commercial sunless tanning products sold in retail outlets.

TABLE 1

| | Formula #25-103- (% w/w) | | | | |
|---|---|---|---|---|---|
| Ingredient | A | B | C | D | E |
| DHA | 5 | 5 | 5 | 5 | 5 |
| Pentylene Glycol | 1 | — | — | — | — |
| 1,3-butylene glycol | — | 1 | — | — | — |
| Hexylene glycol | — | — | 1 | — | — |
| Dimethyl Isosorbide | — | — | — | 1 | — |
| (Arlasolve ® DMI) | | | | | |
| Propylene glycol | — | — | — | — | 1 |
| Citric Acid solution | * | * | * | * | * |
| Water, Deionized | 94 | 94 | 94 | 94 | 94 |

* Solutions are adjusted to a pH of 3.0-3.5

An equal amount of each formula (approximately 150 mg.) from Table 1 was applied to 2″×2″ adjacent sections of forearm, allowed to dry and covered with clothing for the duration of the testing. The application areas were visibly the same color prior to treatment with the solutions and this was confirmed via calorimeter readings. Visual assessment of color development was made at 24 hours post application to record the peak tanning response using a 7-point scale with 0 signifying no color development versus non-treated skin, 3 signifying moderate color development and 6 signifying intensely dark color development. In certain cases the tanning response was also recorded at 72 hours and 5 days post-application to record comparable color intensity and longevity of the tanning response of the formulations. Color development results for sunless tanning solutions from Table 1 are shown in Table 1.1, for Table 2 in Table 2.1 and so forth. In certain cases, both subjective results (e.g., Tables 3.1, 4.1, etc.) and objective results (e.g., Tables 3.2, 4.2, etc.) are shown.

Tables 2 through 5 detail the same humectants and penetrants as used in the formulas from Table 1 at increasing concentration of glycol from 3% up to 20% with the DHA held constant at 5%. The same application and evaluation procedure as described for the formulas in Table 1 was used for the formulations in Tables 2-5.

TABLE 2

| | Formula #25-103- (% w/w) | | | | |
|---|---|---|---|---|---|
| Ingredient | F | G | H | I | J |
| DHA | 5 | 5 | 5 | 5 | 5 |
| Pentylene Glycol | 3 | — | — | — | — |
| 1,3-butylene glycol | — | 3 | — | — | — |
| Hexylene glycol | — | — | 3 | — | — |
| Dimethyl Isosorbide | — | — | — | 3 | — |
| (Arlasolve ® DMI) | | | | | |
| Propylene glycol | — | — | — | — | 3 |
| Citric Acid solution | * | * | * | * | * |
| Water, Deionized | 92 | 92 | 92 | 92 | 92 |

* Solutions are adjusted to a pH of 3.0-3.5

TABLE 3

| | Formula #25-105- (% w/w) | | | | |
|---|---|---|---|---|---|
| Ingredient | A | B | C | D | E |
| DHA | 5 | 5 | 5 | 5 | 5 |
| Pentylene Glycol | 5 | — | — | — | — |
| 1,3-butylene glycol | — | 5 | — | — | — |
| Hexylene glycol | — | — | 5 | — | — |
| Dimethyl Isosorbide | — | — | — | 5 | — |
| (Arlasolve ® DMI) | | | | | |
| Propylene glycol | — | — | — | — | 5 |
| Citric Acid solution | * | * | * | * | * |
| Water, Deionized | 90 | 90 | 90 | 90 | 90 |

* Solutions are adjusted to a pH of 3.0-3.5

TABLE 4

| | Formula #25-105- | | | | |
|---|---|---|---|---|---|
| | (% w/w) | | | | |
| Ingredient | F | G | H | I | J |
| DHA | 5 | 5 | 5 | 5 | 5 |
| Pentylene Glycol | 10 | — | — | — | — |
| 1,3-butylene glycol | — | 10 | — | — | — |
| Hexylene glycol | — | — | 10 | — | — |
| Dimethyl Isosorbide | — | — | — | 10 | — |
| (Arlasolve ® DMI) | | | | | |
| Propylene glycol | — | — | — | — | 10 |
| Citric Acid solution | * | * | * | * | * |
| Water, Deionized | 85 | 85 | 85 | 85 | 85 |

* Solutions are adjusted to a pH of 3.0-3.5

TABLE 5

| | Formula #25-105- | | | | |
|---|---|---|---|---|---|
| | (% w/w) | | | | |
| Ingredient | K | L | M | N | O |
| DHA | 5 | 5 | 5 | 5 | 5 |
| Pentylene Glycol | 20 | — | — | — | — |
| 1,3-butylene glycol | — | 20 | — | — | — |
| Hexylene glycol | — | — | 20 | — | — |
| Dimethyl Isosorbide | — | — | — | 20 | — |
| (Arlasolve ® DMI) | | | | | |
| Propylene glycol | — | — | — | — | 20 |
| Citric Acid solution | * | * | * | * | * |
| Water, Deionized | 75 | 75 | 75 | 75 | 75 |

* Solutions are adjusted to a pH of 3.0-3.5

The tanning results for these simple DHA/glycol solutions listed in Table 1-5 are recorded below. Replicates and triplicates were conducted with several volunteers and average values are recorded. Objective measurements with a Minolta CR-10 Colorimeter modified for skin measurement studies were made in addition to the subjective scoring to clearly document the overall color and hue intensity of the tanning response for those formulas that appeared to give a maximal tanning response:

TABLE 1.1

| | Skin Rating |
|---|---|
| Formula | 24 hrs |
| A | 3.5 |
| B | 1.0 |
| C | 2.0 |
| D | 1.0 |
| E | 2.5 |

TABLE 2.1

| | Skin Rating |
|---|---|
| Formula | 24 hrs |
| F | 2.5 |
| G | 1.0 |
| H | 1.0 |
| I | 0 |
| J | 0 |

TABLE 3.1

| Formula | 24 hrs. | 72 hrs. |
|---|---|---|
| A | 5.0 | 4.0 |
| B | 3.0 | 2.5 |
| C | 4.0 | 2.0 |
| D | 4.0 | 3.0 |
| E | 3.0 | 2.0 |

TABLE 3.2

| Formula | L | a | b |
|---|---|---|---|
| A | 56.1 | 10.8 | 15.8 |
| B | 59.3 | 9.7 | 16.2 |
| C | 59.9 | 9.4 | 13.1 |
| D | 59.6 | 8.7 | 16.9 |
| E | 59.8 | 9.7 | 14.5 |

TABLE 4.1

| Formula | 24 hrs. | 72 hrs. |
|---|---|---|
| F | 6.0 | 4.0 |
| G | 4.0 | 2.0 |
| H | 4.0 | 2.5 |
| I | 4.0 | 1.5 |
| J | 5.0 | 2.0 |

TABLE 4.2

| Formula | L | a | b |
|---|---|---|---|
| F | 54.9 | 11.9 | 17.8 |
| G | 60.0 | 9.6 | 15.2 |
| H | 59.3 | 10.7 | 15.0 |
| I | 57.9 | 10.8 | 13.7 |
| J | 57.4 | 11.6 | 16.1 |

TABLE 5.1

| Formula | 24 hrs. | 5 Days |
|---|---|---|
| K | 4.0 | 3.0 |
| L | 4.0 | 3.0 |
| M | 4.0 | 3.5 |
| N | 4.0 | 2.5 |
| O | 4.0 | 2.5 |

For the results above listed in Tables 1.1 through 5.1, the maximum tanning response for 5% DHA resulted with combinations of pentylene glycol at 5% and 10%. The intensity of the tanning response was greater at 5-10% pentylene glycol than at both the lower and higher concentrations of pentylene glycol tested. Additionally, the tanning response with pentylene glycol and DHA was consistently greater than the tanning response of DHA and the other glycols tested at all concentrations tested, up to the 20% concentration, where the tanning response seemed to level out. Also of note is the observation that formula #25-105M containing 5% DHA and 20% hexylene glycol showed significant cloudiness and precipitation due to limited solubility of the hexylene glycol. Results for this formulation should be discounted.

Overall, these simple solution formulations of 5% DHA and various humectants and penetrants demonstrate an increased tanning response with pentylene glycol over the other humectants and penetrants tested in this series of tests. This advantage is demonstrated in the intensity of the tanning response at peak times (e.g., 24 hours post-application) as well as in the duration of the tanning response over the course of several days.

A second series of glycol humectants and penetrant materials were evaluated at concentrations of 1-20% w/w in combination with 5% DHA w/w; the amount of DHA typically found in commercial sunless tanning products sold in retail outlets. In this series of tests, pentylene glycol was compared to isoprene glycol (isopentyl diol; Barnet), ethoxydiglycol, MP Diol (2-methyl-1,3-propanediol; Lyondell), and glycerin.

This series of experiments evaluated the aforementioned glycol humectant and penetrant materials at 1%, 3%, 5%, 10%, and 20% w/w in combination with 5% w/w DHA in aqueous solutions.

For illustration purposes, Table 6 lists the glycol humectant and penetrant materials at 5% w/w in combination with 5% w/w DHA.

An equal amount of each formula (approximately 150 mg.) from Table 6 was applied to 2″×2″ adjacent sections of the volar forearm, allowed to dry, and covered with clothing for the duration of the testing. The application areas were visibly the same color prior to treatment with the solutions and this was confirmed via colorimeter readings.

TABLE 6

| | Formula #25-153- (% w/w) | | | | |
|---|---|---|---|---|---|
| Ingredient | A | B | C | D | E |
| DHA | 5 | 5 | 5 | 5 | 5 |
| Pentylene Glycol | 5 | — | — | — | — |
| Isoprene Glycol (Barnet) | — | 5 | — | — | — |
| Ethoxydiglycol (Trivalin SF; Tri-K) | — | — | 5 | — | — |
| MP Diol (Lyondell) | — | — | — | 5 | — |
| Glycerin | — | — | — | — | 5 |
| Citric Acid solution | * | * | * | * | * |
| Water, Deionized | 90 | 90 | 90 | 90 | 90 |

* Solutions are adjusted to a pH of 3.0-3.5

The tanning response results of the formula solutions from Table 6 are detailed below in Tables 6.1 Replicates and triplicates were conducted with several volunteers and average values are recorded. Visual assessment of color development was made at 24 hours post application to record the peak tanning response using a 7-point scale with 0 signifying no color development versus non-treated skin, 3 signifying moderate color development and 6 signifying intensely dark color development. The tanning response was also recorded at 5 days post-application to record comparable color intensity and longevity of the tanning response of the formulations. Objective measurements with a Minolta CR-10 Colorimeter modified for skin measurement studies were selectively made in addition to the subjective scoring to clearly document the overall color and hue intensity of the tanning response.

TABLE 6.1

| Formula | 24 hrs. | 5 Days |
|---|---|---|
| A | 4.5 | 3.5 |
| B | 3.5 | 2.5 |
| C | 3.5 | 2.5 |
| D | 4.5 | 3.0 |
| E | 4.5 | 2.5 |

Results from Tables 6.1 demonstrate the most intense, longest lasting tanning response of all the formulas tested in this series showing a peak response for 5% w/w pentylene glycol and 5% w/w DHA. In general, the intensity of the tanning response for combinations of DHA and pentylene glycol was greater than the other glycols tested at any concentration. Note that formulas containing 10% and 20% of MP Diol, respectively, were cloudy demonstrating incomplete solubility of MP Diol and results of these experiments should be discounted.

A third series of experiments was conducted to determine the relative tanning response of aqueous formulations containing DHA and pentylene glycol when the cocentration of DHA and pentylene glycol were varied between ratios of 1:4 and 4:1. For this series of experiments, DHA concentration varied between 2.5-10% and pentylene glycol varied between 1.25-20% as shown in Table 7.

TABLE 7
(1:4-4:1 DHA/1,2,-Pentanediol Ratios)

| | | |
|---|---|---|
| Series | % DHA | % 1,2-Pentanediol |

| | | | | | |
|---|---|---|---|---|---|
| 1 | 2.5 | 10.0 | 5.0 | 2.5 | 1.25 |
| 2 | 5.0 | 20.0 | 10.0 | 5.0 | 2.50 |
| 3 | 7.5 | 15.0 | 7.5 | 3.75 | 1.88 |
| 4 | 10.0 | 20.0 | 10.0 | 5.0 | 2.5 |

All of the various ratios of DHA and pentylene glycol were converted into simple aqueous solutions, pH adjusted to 3.0-3.5 and evaluated for tanning response. Again, for illustration purposes, Table 8 shows the series of pentylene glycol ratios tested in combination with 10% w/w DHA.

TABLE 8

| | Formula #25-179-(% w/w) | | | |
|---|---|---|---|---|
| Ingredient | A | B | C | D |
| DHA | 10 | 10 | 10 | 10 |
| Pentylene Glycol (Hydrolite-5) | 20.0 | 10.0 | 5.0 | 2.5 |
| Citric Acid solution | * | * | * | * |
| Water, Deionized | 70 | 80 | 85 | 88.5 |

*Solutions are adjusted to a pH of 3.0-3.5

An equal amount of each formula (approximately 150 mg.) from Table 8 was applied to 2″×2″ adjacent sections of forearm, allowed to dry and covered with clothing for the duration of the testing. The application areas were visibly the same color prior to treatment with the solutions and this was confirmed via calorimeter readings. Visual assessment of color development was made at 24 hrs, 48 hrs or 72 hours and at 5 days post application to record the tanning response intensity and durability using a 7-point scale with 0 signifying no color development versus non-treated skin, 3 signifying moderate color development and 6 signifying intensely dark color development. Objective measurements with a Minolta CR-10 Colorimeter modified for skin measurement studies were made in addition to the subjective scoring to clearly document the overall color and hue intensity of the tanning response for those formulas that appeared to give a maximal tanning response:

Color development scores for sunless tanning solutions from Table 8 are shown in Tables 8.1 and 8.2 below:

TABLE 8.1

| Formula | 24 hrs. | 72 hrs | 5 Days |
|---|---|---|---|
| A | 5.0 | 5.0 | 2.5 |
| B | 6.0 | 5.0 | 4.0 |
| C | 5.0 | 5.0 | 2.5 |
| D | 5.0 | 5.0 | 3.0 |

TABLE 8.2
(5-day results)

| Formula | L | a | b |
|---|---|---|---|
| A | 61.9 | 11.3 | 14.1 |
| B | 60.1 | 12.4 | 15.2 |
| C | 61.1 | 12.0 | 13.5 |
| D | 60.8 | 11.8 | 13.3 |

For all the formula combinations derived from Table 7, the most intense tanning response was seen with combinations of pentylene glycol and 10% DHA(Table 8) with tanning results recorded in Tables 8.1 and 8.2 above.

Typically, sunless tanning products containing DHA produce a malodor when applied to the skin. This malodor is characterized as a “burnt caramel” or “burnt sugar” smell and is generally rated as unpleasant by consumers using sunless tanning products. Typically, this malodor increases with increased levels of DHA in the formula. Automated sunless tanning spray formulas typically contain high levels of DHA between 7-12% and have been shown to produce this DHA malodor in use. Sunless tanning solutions containing pentylene glycol and DHA as described in the various examples above were found to be virtually odorless when applied to the skin and remained odorless throughout the evaluations. In order to evaluate the consumer perception of automated sunless tanning spray formulations containing DHA and pentylene glycol, a consumer test of two test formulations, shown in Table 9 and 10 below, was undertaken.

TABLE 9
Sunless Tanning Solution for Automatic Misting
Test Formula # 25-67

| Ingredient | % W/W |
|---|---|
| Simethicone | 0.01 |
| Dihydroxyacetone | 10.00 |
| Pentylene Glycol | 5.00 |
| Butylene Glycol | 1.20 |
| Polysorbate 20 | 0.60 |
| Citric Acid Sol'n 25% | 0.22* |
| FD&C Yellow # 5 (50:50 water/butylenes glycol) 1% | 1.76 |
| FD&C Red # 40 (50:50 water/butylenes glycol) 1% | 2.04 |
| FD&C Red # 33 (50:50 water/butylenes glycol) 2.5% | 0.41 |
| FD&C Green # 5 (50:50 water/butylenes glycol) 1% | 3.50 |
| Sodium Citrate Solution 25% | 0.14** |
| Phenoxyethanol | 0.30 |
| Aloe Vera Gel, concentrate | 0.10 |
| Water, Deionized, q.s. a.d. | 100.00 |

*q.s. to pH 2.60
**q.s. to pH 3.20

TABLE 10
Sunless Tanning Solution for Automatic Misting
Control Formula # 24-119B

| Ingredient | % W/W |
|---|---|
| Water and stabilized Aloe Vera Gel | 69.89 |
| Ethoxydiglycol | 1.00 |
| Dihydroxyacetone | 10.00 |
| Glycerin | 5.00 |
| Sorbitol Solution 70% | 2.00 |
| Polysorbate 20 | 0.50 |
| Citric Acid Sol'n 25% | 1.26* |
| FD&C Yellow # 5 (50:50 water/butylenes glycol) 1% | 1.75 |
| FD&C Red # 40 (50:50 water/butylenes glycol) 1% | 2.04 |
| FD&C Red # 33 (50:50 water/butylenes glycol) 2.5% | 0.51 |
| FD&C Green # 5 (50:50 water/butylenes glycol) 1% | 3.50 |
| Sodium Citrate Solution 25% | 1.35** |
| DMDM Hydantoin | 0.20 |
| Alcohol SDA 40, 200 Proof | 1.00 |

*q.s to pH 2.60
**q.s. to pH 3.20

Formula #25-67 containing 5% pentylene glycol and 10% DHA detailed above was evaluated by 39 consumers who were regular users of an automated sunless tanning spray mist product containing 10% DHA but no pentylene glycol. This control formula is listed as Formula #24-119B, also detailed above. The consumers in this test had typically received a sunless tanning spray treatment with the control product 3-4 times per month for the past 12-24 months. The consumers were asked to evaluate a new sunless tanning automatic spray solution (Test Formula #25-67), and, after receiving the application, to rate the test product for odor, tan longevity (time to fade) and product affect on skin texture. A randomized design for this test involved the use of the test formula followed by the control formula by approximately half of the test subjects and the use of the control formula followed by the test formula by the other half of the test subjects. Consumers in this test rated the Test Formula's affect on skin texture as the same as the control. However, the tan received from the Test product was rated somewhat darker and, directionally, lasting longer than the control formula.

Consumers rated the odor of the Test Formula as having significantly less odor than the Control formula with approximately 85% of the consumers rating the formula with pentylene glycol as much less/somewhat less, approximately 13% rating both formulas the same, and approximately 3% rating the Test formula as having somewhat more odor. These results were highly significant and demonstrate the ability of pentylene glycol to enhance the tanning response of DHA and produce a virtually odorless automated sunless tanning spray.

Although not listed in the above sunless tanning formulations, it is anticipated that the aqueous formulations containing DHA and pentylene glycol may also contain acidifying agents, alkalizing agents, antimicrobial agents, antioxidants, botanicals, buffering agents, chelating agents, other coloring additives, cosmeceuticals, defoaming agents, dermatologically active agents, dispersing agents, emollients, emulsifying agents, humectants, fragrances, moisturizers, other preservatives, sugars, sunscreen agents, surfactants, suspending agents, thickening agents, and vitamins.

While the examples set forth above illustrate specific embodiments of the invention and should be considered non-limiting examples with variations and modifications thereof all being within the spirit and purview of this invention.

1. A cosmetic sunless tanning spray formulation which produces a darker, longer-lasting and virtually odorless tan, said formulation comprising:
a) 1%-20% 1,2-pentanediol,
b) 0.5%-20%Dihydroxyacetone (DHA), and
c) an aqueous vehicle

2. The cosmetic formulation of claim 1 wherein the pH is maintained between 3.0-6.0

3. The cosmetic formulation of claim 1 wherein the pH is maintained between 3.0-4.0 with a buffer system

4. The cosmetic formulation of claim 1 where the ratio of DHA to 1,2-pentanediol is between 1:4 and 4:1.

5. The cosmetic formulation of claim 1 containing 5-10% 1,2-pentanediol and 10% DHA

6. The cosmetic formulation of claim 5 wherein the pH is maintained between 3.0-6.0

7. The cosmetic formulation of claim 5 wherein the pH is maintained between 3.0-4.0 with a buffer system

8. The cosmetic formulation of claim 1, wherein the carrier vehicle contains one or more ingredients selected from the group consisting of water soluble or water dispersible acidifying agents, alkalizing agents, aerosol propellants, antimicrobial agents, antioxidants, buffering agents, chelating agents, coloring additives, defoaming agents, dermatologically active agents, dispersing agents, emollients, emulsifying agents, humectants, fragrances, moisturizers, preservatives, solvents, sugars, sunscreen agents, surfactants, suspending agents, thickening agents, and vitamins

9. The cosmetic formulation of claim 5, wherein the carrier vehicle contains at least one ingredient selected from the group consisting of water soluble or water dispersible acidifying agents, alkalizing agents, aerosol propellants, antimicrobial agents, antioxidants, buffering agents, chelating agents, coloring additives, defoaming agents, dermatologically active agents, dispersing agents, emollients, emulsifying agents, humectants, fragrances, moisturizers, preservatives, solvents, sugars, sunscreen agents, surfactants, suspending agents, thickening agents, and vitamins
